(19) United States
(12) Patent Application Publication
(10) Pub. No.: US 2023/0241335 A1
(43) Pub. Date: Aug. 3, 2023

(54) ST LIGHT ENGINE ASSEMBLY FOR USE WITH AN OXYGEN CONCENTRATOR AND METHOD OF ASSEMBLING SAME

(71) Applicant: Smart Tubing LLC, Las Vegas, NV (US)

(72) Inventors: Kristen Abbott, Las Vegas, NV (US); Frank Abbott, JR., Las Vegas, NV (US); Frank Abbott, III, Las Vegas, NV (US); Pierre Gross, Las Vegas, NV (US)

(21) Appl. No.: 18/102,585

(22) Filed: Jan. 27, 2023

Related U.S. Application Data
(60) Provisional application No. 63/304,364, filed on Jan. 28, 2022.

(51) Int. Cl. A61M 16/00; A61M 16/10; A61M 16/08

(52) U.S. Cl. CPC A61M 16/0003; A61M 16/101; A61M 16/0858; A61M 2205/587

(57) ABSTRACT

A lighted air supply delivery assembly for use in delivering air to a patient is described herein. The lighted air supply delivery assembly includes an air/light connection tubing coupled between an oxygen concentrator and a patient oxygen delivery. The air/light connection tubing defines an airflow chamber for delivering a flow of oxygenated air from the oxygen concentrator to the patient oxygen delivery assembly, and includes one or more optical fibers positioned within the airflow chamber. A fiber-optic light engine coupled to the one or more optical fibers. A controller is coupled to the fiber-optic light engine and includes a processor programmed to execute algorithm steps of operating the fiber-optic light engine to display a light visible through the air/light connection tubing via the one or more optical fibers.

### CROSS-REFERENCE TO RELATED APPLICATIONS

This application claims benefit of U.S. Provisional Patent Application Ser. No. 63/304,364 filed Jan. 28, 2022, the disclosure of which is hereby incorporated by reference in its entirety.

### BACKGROUND OF THE INVENTION

An oxygen concentrator is a medical device that provides extra oxygen to a patient. The doctor may prescribe an oxygen concentrator to a patient who has a health condition that causes oxygen levels to drop too low. Oxygen concentrators help patients who have trouble breathing due to conditions such as asthma.

Oxygen concentrators include tubes that provide oxygen to the patient. These tubes sometimes get wrapped around pieces of furniture causing a tripping hazard to the patient, caretaker, doctor, etc. In addition, when trying to move the oxygen concentrator, the tubing may get in the way of the person moving the equipment. When providing oxygen to a patient, the tubes will sometimes get stuck on a chair or tied in a knot which inhibits the flow of air to the patient.

Recent studies have shown that a build-up of impurities in the tubes carrying oxygen from the concentrator to the patient causes respiratory infections. An oxygen concentrator apparatus designed to overcome one or more of the aforementioned challenges is desired.

### BRIEF SUMMARY OF THE INVENTION

In one aspect of the present invention, a lighted air supply delivery assembly for use in delivering air to a patient is provided. The lighted air supply delivery assembly includes an air/light connection tubing coupled between an oxygen concentrator and a patient oxygen delivery. The air/light connection tubing defines an airflow chamber for delivering a flow of oxygenated air from the oxygen concentrator to the patient oxygen delivery assembly, and includes one or more optical fibers positioned within the airflow chamber. A fiber-optic light engine coupled to the one or more optical fibers. A controller is coupled to the fiber-optic light engine and includes a processor programmed to execute algorithm steps of operating the fiber-optic light engine to display a light visible through the air/light connection tubing via the one or more optical fibers.

In another aspect of the present invention, an oxygen delivery system for delivering oxygenated air to a patient is provided. The oxygen delivery system includes an oxygen concentrator, a patient oxygen delivery assembly adapted to be mounted to a patient's face, and a lighted air supply delivery assembly coupled between the oxygen concentrator and the patient oxygen delivery assembly. The lighted air supply delivery assembly includes an air/light connection tubing defining an airflow chamber for delivering a flow of oxygenated air from the oxygen concentrator to the patient oxygen delivery assembly, and including one or more optical fibers positioned within the airflow chamber. A fiber-optic light engine coupled to the one or more optical fibers. A controller is coupled to the fiber-optic light engine and includes a processor programmed to execute algorithm steps of operating the fiber-optic light engine to display a light visible through the air/light connection tubing via the one or more optical fibers.

In yet another aspect of the present invention, a method of assembling a lighted air supply delivery assembly for use in delivering air to a patient is provided. The method includes providing an air/light connection tubing defining an airflow chamber for delivering a flow of oxygenated air from an oxygen concentrator to a patient oxygen delivery assembly, and including one or more optical fibers positioned within the airflow chamber. The method also includes coupling a fiber-optic light engine to the one or more optical fibers, coupling an air pressure sensor for measuring an air pressure to the an air/light connection tubing, and coupling a controller to the fiber-optic light engine and the air pressure sensor. The controller including a processor programmed to execute algorithm steps of operating the fiber-optic light engine to display the light visible through the air/light connection tubing based on the measured air pressure.

### BRIEF DESCRIPTION OF THE DRAWINGS

Other advantages of the present invention will be readily appreciated as the same becomes better understood by reference to the following detailed description when considered in connection with the accompanying drawings.

FIGS. 1 and 2 are schematic diagrams of a patient oxygen delivery system;

FIGS. 3-6 are schematic illustrations of a lighted air supply delivery assembly that may be used with the patent oxygen delivery system shown in FIGS. 1 and 2;

FIGS. 7 and 8 are schematic cross-sectional views of an air/light connection tubing that may be used with the lighted air supply delivery assembly;

FIGS. 9-12 are schematic perspective views of a main housing suitable for use with the lighted air supply delivery assembly;

FIGS. 13 and 14 are top views of a portion of the lighted air supply delivery assembly;

FIG. 15 is a schematic illustration of an electrical circuit diagram of a controller that may be used with the lighted air supply delivery assembly; and

FIGS. 16 and 17 are flowcharts illustrating algorithms used during operation of the lighted air supply delivery assembly.

Corresponding reference characters indicate corresponding parts throughout the drawings.

### DETAILED DESCRIPTION OF THE INVENTION

With reference to the drawings and in operation, the present invention is directed towards an oxygen delivery system that includes an oxygen concentrator apparatus and lighted air supply delivery assembly for delivering oxygenated air from the oxygen concentrator apparatus to a patient. The lighted air supply delivery assembly includes a ST Main Housing including a ST Light Engine housed in a cylindrical cavity assembly within an aluminum housing, a quick connect fiber optic twist connector, a pressure sensor, a Main circuit board, a Bluetooth™ circuit board, two brass lube connectors with tapered threads, a heat sink machined into the aluminum housing for cooling the Light Engine, a circuit board on which the pressure sensor and other logic chips are mounted, a 12-volt barrel shaped electrical connection point, an on/off button switch, an oxygen chamber to allow oxygen to flow into and out of the apparatus, a 90-degree elbow to connect the oxygen chamber to the pressure sensor via a small plastic tube, a buzzer that can become audible when a signal is received from a logic chip mounted on the Main circuit board, a logic chip that can command the light engine to turn on and off repeatedly, a top cover made of aluminum that has some interior machined cavities above the various components including above the Main circuit board and red and blue flashing lights on the Bluetooth circuit board to indicate that the device is ready to connect via Bluetooth to: a smart phone, tablet. computer, iPad, or any other electronic Bluetooth enabled device.

Referring now to the drawings, wherein like numerals indicate like parts throughout the several views, the present disclosure is generally directed toward an oxygen delivery system 10 that includes an oxygen concentrator apparatus and lighted air supply delivery assembly for delivering oxygenated air from the oxygen concentrator apparatus to a patient. The oxygen concentrator apparatus may concentrate oxygen out of an air stream to provide oxygen gas to a user. The oxygen concentrator apparatus may be a standalone device or a portable oxygen concentrator. For example, the portable oxygen concentrators may have a weight and size that allows the oxygen concentrator to be carried by hand and/or by a carrying case. When in use, air enters the oxygen concentrator and is compressed. The compressed air is then forced into a canister before making its way to a patient via flexible transparent tubing.

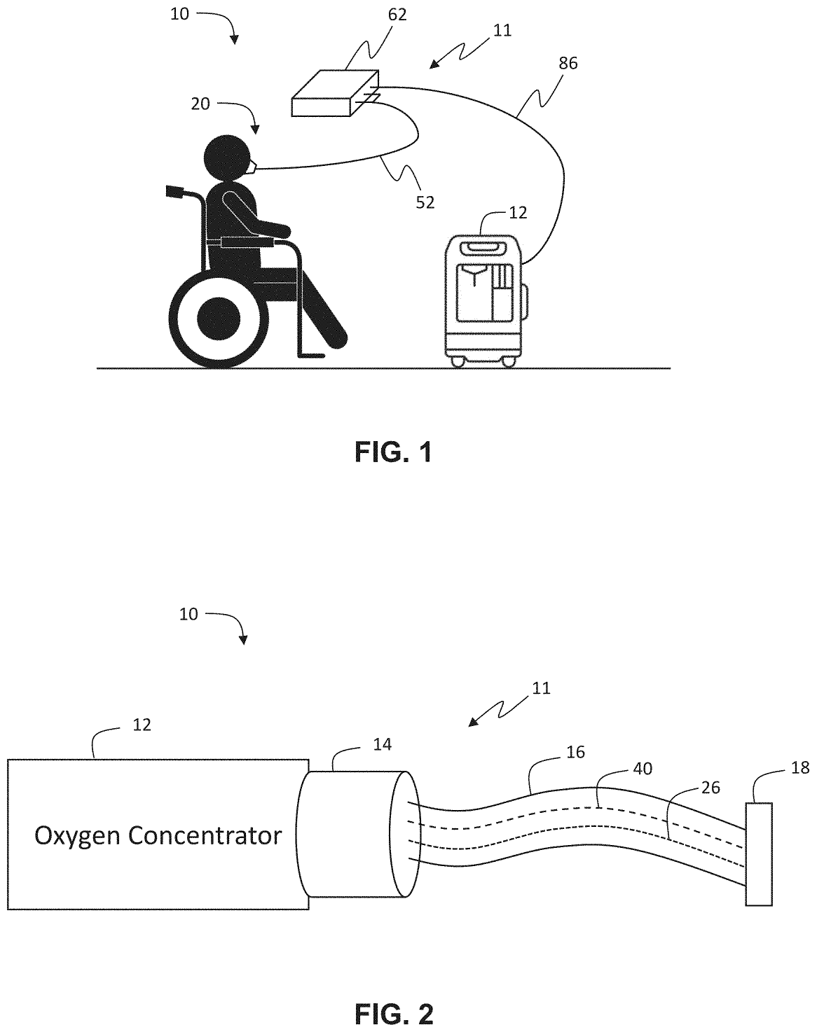

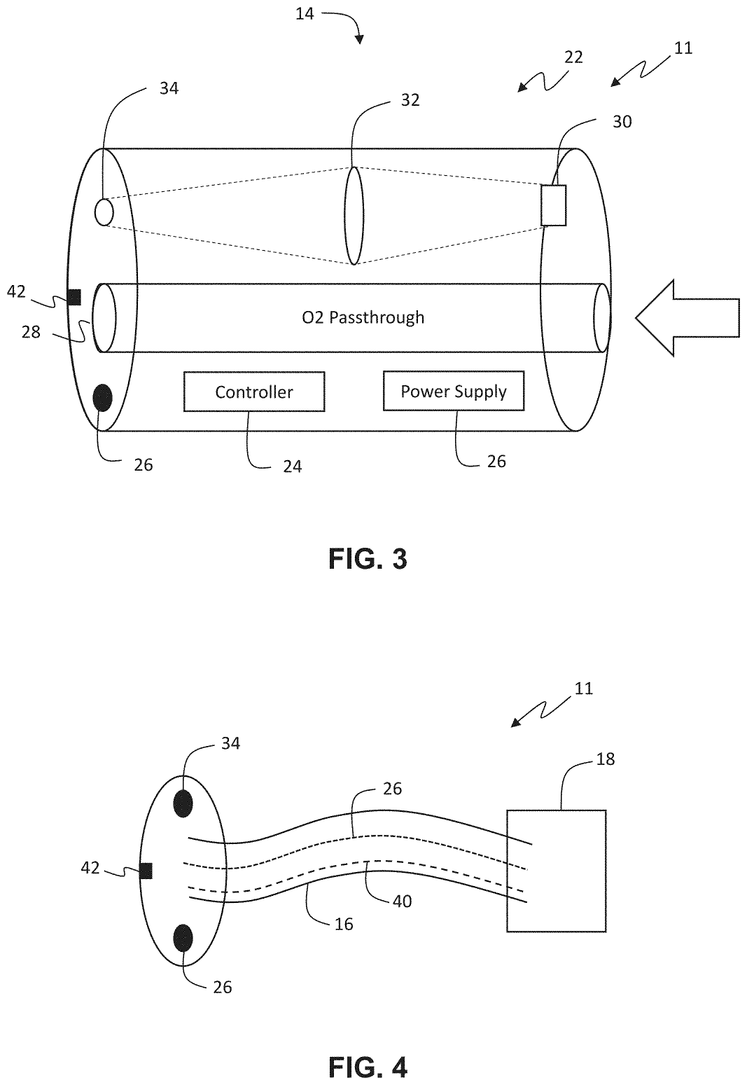

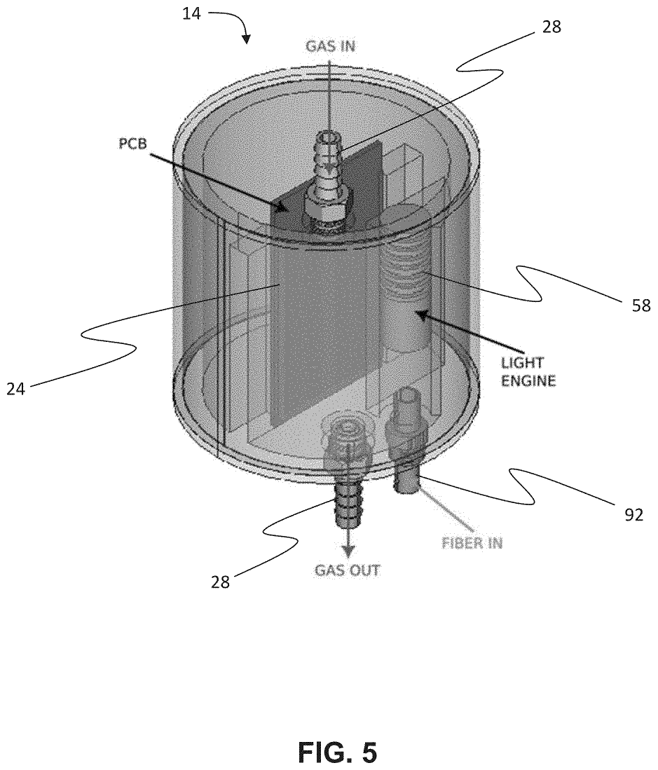

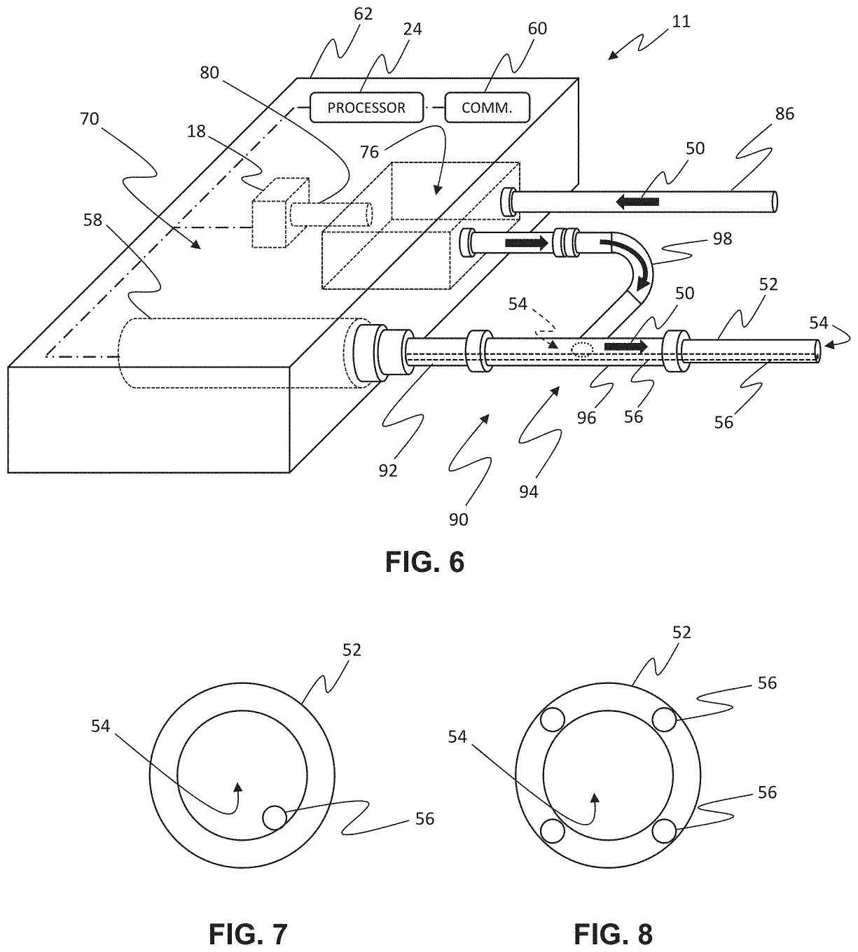

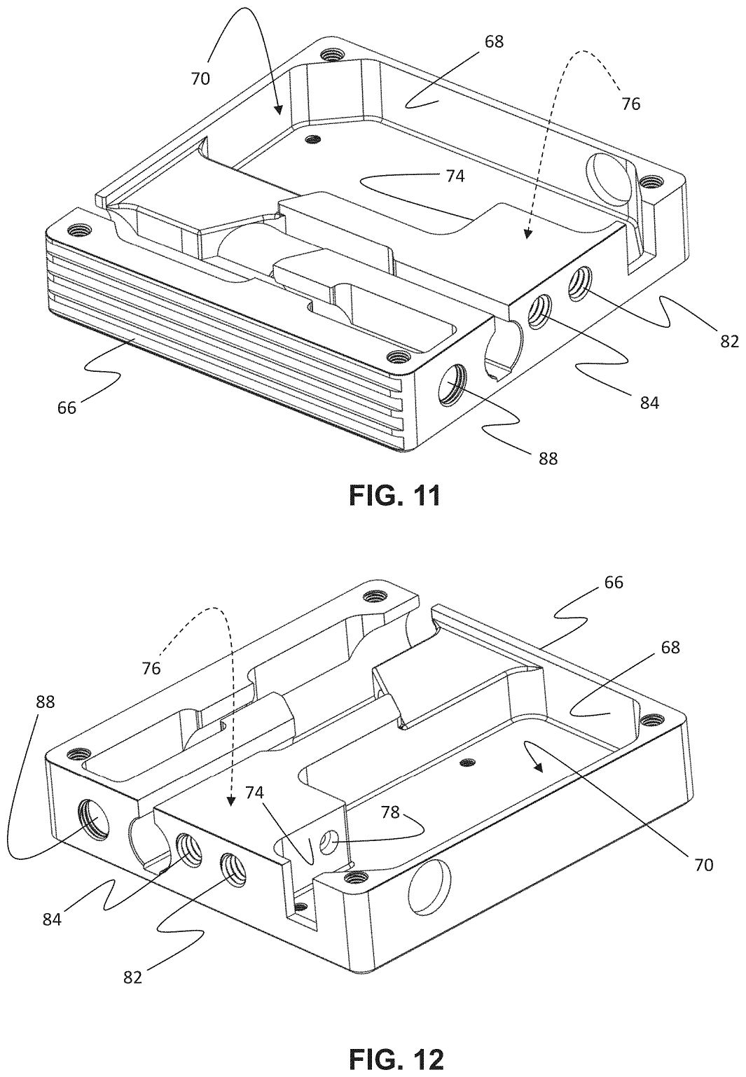

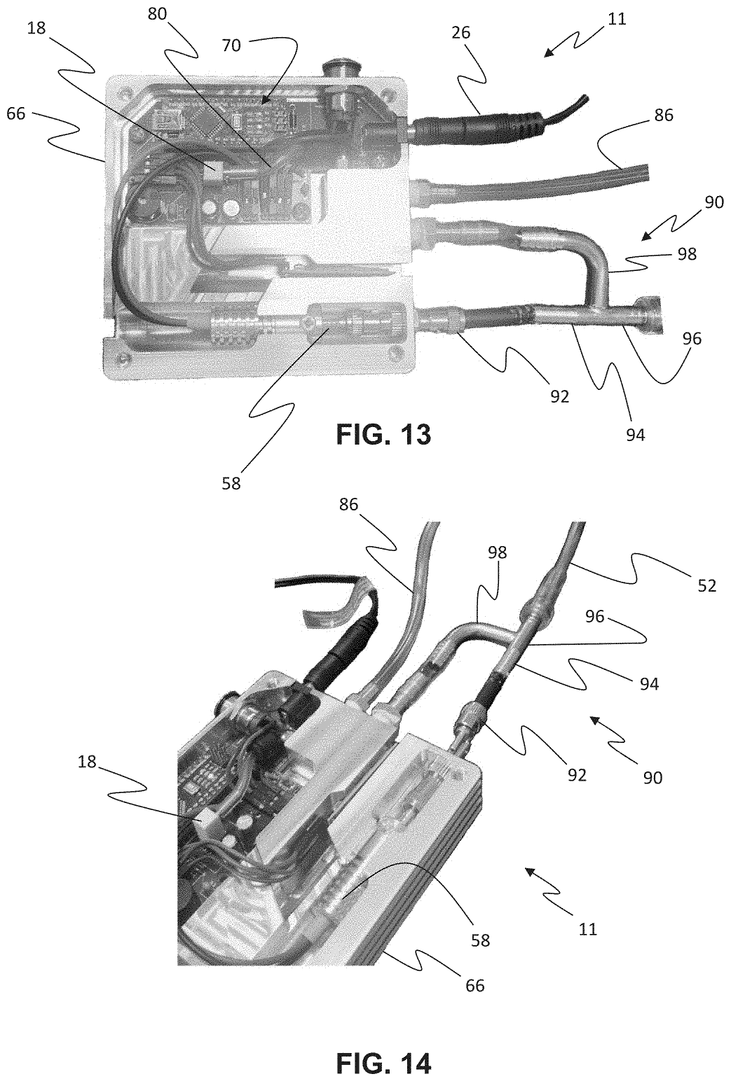

Referring to FIGS. 1-15, in the illustrated embodiment, the oxygen concentrator and related ST Main Housing apparatus are operable by a user for providing oxygen to a patient. The oxygen delivery system 10 includes a lighted air supply delivery assembly 11 and an oxygen concentrator 12. The lighted air supply delivery assembly 11 includes a ST Main Housing and ST light engine assembly 14, a flexible tube 16, a sensor 18, and a patient oxygen delivery assembly 20. The patient oxygen delivery assembly 20 may include a nasal cannula, a face mask, and/or any suitable apparatus for delivering oxygenated air to a patient.

The ST Main Housing and ST Light Engine assembly 14 includes a cylindrical housing with an oxygen passthrough defined through the center of the housing. A set screw is used to secure the fiber optic cable within the ST Main Housing containing the ST light engine assembly 14. A flexible oxygen tube is used to connect the Main Housing to the output of the oxygen concentrator 12. A blue or green LED chip is located within the light engine against the LED chip. A lens is defined within the housing to focus the light to a point in the middle of the light engine output. A fiber optic connector is located within and outside the light engine. The ST Main Housing of the ST light engine assembly 14 also includes a 12 volt input barrel connection point for the purpose of bringing power to the Main Housing. A logic chip mounted on the Main circuit board will change the steady glow of the LED to a flashing light engine output based on the data it receives from the sensors located on the Main circuit board within the Main Housing (e.g., low air pressure is sensed within the Main housing when a blockage is detected via the on board pressure sensor. A signal is sent to the LED Light engine to change steady color of light output to a flashing light output).

The ST light engine assembly 14 is coupled between the oxygen concentrator output 12 and an oxygen tube that leads to the user of oxygen. The ST light engine assembly 14 is configured to notify a user of an interruption in the flow of oxygen being delivered to the patient from the oxygen concentrator 12. The Main Housing and ST light engine within may be coupled to the oxygen concentrator 12 via a set screw or similar coupling device, Velcro or may be suspended via cable or other flexible hanging material. The Main Housing of the ST light engine assembly 14 may be cylindrical and have a hard housing, or rectangular in shape.

The ST light engine assembly 14 includes a housing enclosing a light assembly 22, a controller 24, a power supply 26, and an oxygen passthrough line 28. The light assembly 22 includes an LED light 30, a lens 32, and a fiber optic output 34. The light assembly 22 emits a light from the LED light 30 that may pass through the lens 32 to the fiber optic output 34. The LED light 30 is configured to flash on and off based on the data received from the sensor 18 based on the amount of oxygen being provided to the patient. The lens 32 is configured to focus the LED light 30 to a point in the middle of the ST light engine assembly 14 to the fiber optic output 34. The fiber optic output 34 may be located in the light assembly 22 or cylindrical housing.

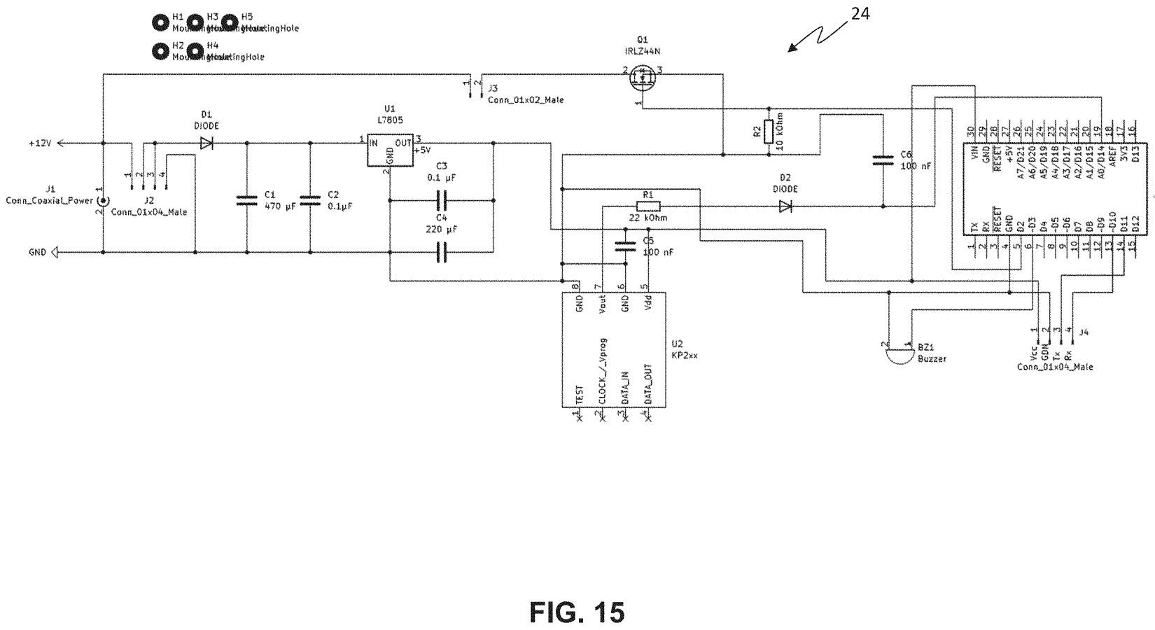

The controller 24 may be a green or blue LED chip. The logic chip that controls the LED Light engine is configured to change the steady color of light to a flashing color of LED light 30. For example, the LED green or blue color light is configured to flash the color of the LED light 30 based on the data it receives from the pressure sensor 18 via a logic chip which is located on the Main circuit board. In some embodiments, the controller 24 may include the electrical circuit diagram shown in FIG. 15.

The external 12 volt power supply 26 is configured to power the electronics in the main housing and the ST light engine assembly 14. For example, the power supply 26 is configured to power the ST light engine assembly 14 in receiving information from the sensor 18 that the back pressure or oxygen flow rate is below a predetermined level and in response change the steady color of the LED light 30 to the flashing color depending on the level of back pressure measured. In various embodiments, the oxygen delivery system 10 uses an inter-integrated circuit (I2C) interface to power the pressure sensor on the main circuit board and the LED Light engine. For example, when in use, the LED light 30 will light up blue or green when the oxygen level is within a predetermined level. However, if the oxygen level is below the predetermined level of oxygen, the sensor will detect the low oxygen level and change the LED light 30 from a steady glow to a flashing light output.

The oxygen passthrough 28 defines a cavity 36 within the ST light engine assembly 14 to allow the oxygen to go through the main housing of the ST light engine assembly 14.

The flexible tube 16 defines a cavity 38. The flexible tube 16 is coupled to the main housing of the ST light engine assembly 14. The flexible tube 16 and the main housing of the ST light engine assembly 14 may be coupled to one another via a threaded strain relief connection or push-on standard medical oxygen tube connectors. The flexible tube 16 includes a side glow fiber optic cable 40 disposed within the cavity 38. In another embodiment, the side glow fiber optic cable 40 may be one of the sides of the flexible tube 16.

The main housing of the ST light engine assembly 14 and the flexible tube 16 may include a notch 42, 44. The notch 42 on the ST light engine assembly 14 will line up with the notch 44 on the flexible tube 16. By lining up the notch 42 on the ST light engine assembly 14 with the notch 44 on the flexible tube 16, and the I2C connector will be lined up as well as the fiber optic output 34 will be lined up to the fiber optic cable 40, described herein.

In one embodiment, the fiber optic cable 40 or side-glow fiber optic cable may be extruded in the flexible tube 16.

In another embodiment, the ST light engine assembly 14 and flexible tube 16 may be coupled via a compression fit with a collar that screws on to the threads at the bottom of the ST light engine assembly 14 and the top of the flexible tube 16.

In yet another embodiment, the flexible tube 16 may be retractable into the oxygen concentrator 12 of the oxygen delivery system 10. By retracting back into the oxygen concentrator 12, the flexible tube 16 would be out of the way of a patient or person when not in use. By retracting back into the oxygen concentrator 12, many hazards may be avoided such as tripping, catching on doors, tables, etc. In addition, by retracting the flexible tube 16 the patient may avoid tubing kinks, wrapping the tubing around the patient's neck, etc. In various embodiment, the flexible tube 16 may be anywhere from 25 to 150 feet long.

The sensor 18 is mounted on the main circuit board within the main housing. The sensor 18 is configured to determine an amount of back pressure that will indicate that oxygen is being provided to the patient, or the contrary, the flow of oxygen has been curtailed to the user. The sensor is powered by electricity being supplied to the main circuit board within the main housing of the ST light engine assembly.

The logic chip controllers 24 include one or more processors operable to execute program instructions in memory. The program instructions are operable to perform various predefined methods that are used to operate the components contained within the main housing of the ST light engine assembly. The controller 24 may include program instructions for operating the oxygen concentrator 12, the ST light engine assembly 14, and the pressure sensor. In various embodiments, the controller 24 includes the processor programmed to detect an interruption in the supply of oxygen to the patient via the flexible tube 16 and flash the selected color of light presented by the LED light 30 and the fiber optic cable 40 upon detecting the amount of oxygen being provided to the patient has fallen below a predetermined value. The processor includes a microcontroller included on a circuit board disposed in the ST light engine assembly 14. The processor is coupled to various components of the oxygen delivery system 10 including, but not limited to, the ST light engine assembly 14 to control any electrical component. In another example, the controller 24 is programmed to monitor the sensor 18 for the levels of oxygen being provided to the patient. If the sensor 18 is triggered, the ST light engine assembly 14 is enabled to change the color of the LED light 30 which changes the color of the fiber optic cable 40 attracting a user to assist the patient.

In some embodiments, the oxygen delivery system 10 includes an ST light engine assembly 14 coupled between an oxygenator 12 and nasal cannula 20. The ST light engine assembly 14 is configured to notify a user of a flow rate of oxygenated air being provided to the patient. The ST light engine assembly 14 includes a light assembly 22 including an LED light 30, a lens 32, a fiber optic output 34, a power supply 26, a controller 24, and an oxygen passthrough line 28. A flexible tube 16 is coupled between the light assembly 22 and the nasal cannula 20 and includes a fiber optic cable 40 disposed within the flexible tube 16. A sensor 18 is coupled to the flexible tube 16. The sensor 18 is configured to determine a flow rate of oxygenated air being provided to the patient via the nasal cannula 20. A controller 24 includes a processor programmed to change the color of the LED light 30 upon detecting a change in the flow rate of oxygen being provided to the patient falling below a predetermined value.

Referring to FIGS. 1 and 6-15, in some embodiments, the oxygen delivery system 10 includes an oxygen concentrator 12, a patient oxygen delivery assembly 20 adapted to be mounted to a patient's face, and a lighted air supply delivery assembly 11 that is coupled between the oxygen concentrator 12 and the patient oxygen delivery assembly 20 for delivering a flow of oxygenated air 50 from the oxygen concentrator 12 to the patient oxygen delivery assembly 20.

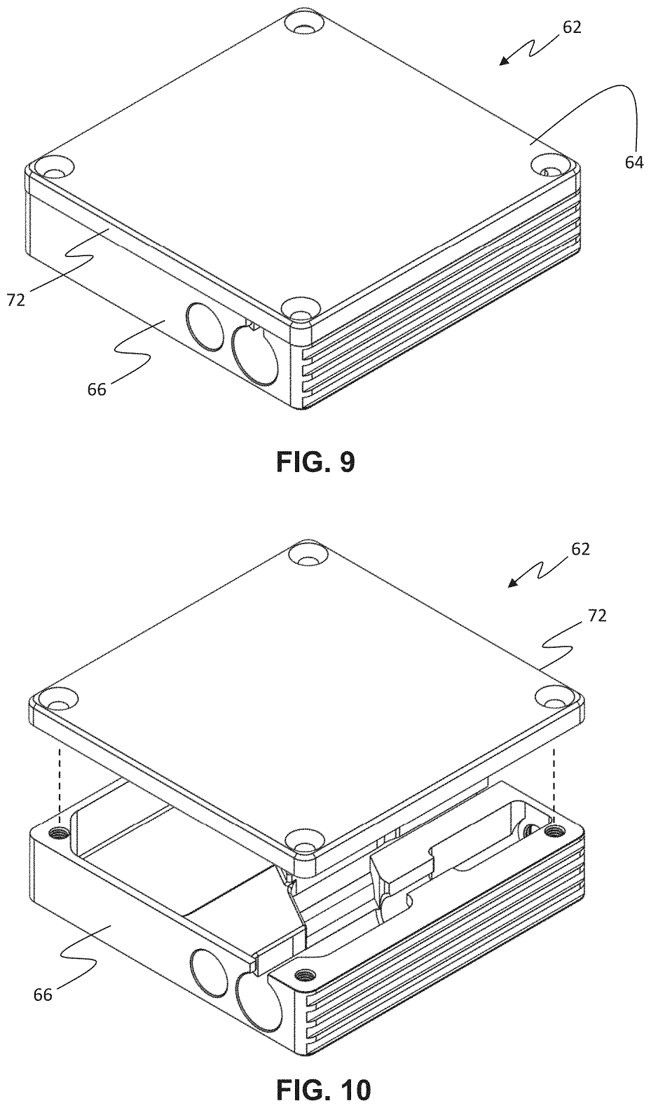

In the illustrated embodiment, the lighted air supply delivery assembly 11 includes an air/light connection tubing 52 defining an airflow chamber 54 for delivering a flow of oxygenated air 50 from the oxygen concentrator 12 to the patient oxygen delivery assembly 20. The air/light connection tubing 52 also includes one or more optical fibers 56 positioned within the airflow chamber 54 and extending along a length of the air/light connection tubing 52 for displaying a light visible through the air/light connection tubing 52. In some embodiments, as shown in FIG. 7, the optical fibers 56 positioned within the airflow chamber 54 and adjacent to an inner surface of the air/light connection tubing 52. In other embodiments, as shown in FIG. 8, optical fibers 56 may be embedded within a tube wall of the air/light connection tubing 52.

The lighted air supply delivery assembly 11 also includes a fiber-optic light engine 58 that is coupled to the optical fibers 56 and a controller 24 that includes a processor that is coupled to the fiber-optic light engine 58 and programmed to execute algorithm steps of operating the fiber-optic light engine 58 to display a flashing light visible through the air/light connection tubing 52 via the optical fibers 56.

The lighted air supply delivery assembly 11 may also include an air pressure sensor 18 that is configured to measure an air pressure within the lighted air supply delivery assembly 11. The processor of the controller 24 may be programmed to execute algorithm steps of operating the fiber-optic light engine 58 to display the flashing light that is visible through the air/light connection tubing 52 via the optical fibers 56 based on the measured air pressure. For example, in some embodiments, the controller 24 may receive a signal from the air pressure sensor 18 indicated a reduction and/or loss of air pressure within the lighted air supply delivery assembly 11, and operate the fiber-optic light engine 58 to display a flashing light via the optical fibers 56 along a length of the air/light connection tubing 52 to facilitate notifying a care giver of the reduction and/or loss of air pressure.

In some embodiments, the lighted air supply delivery assembly 11 may also include a wireless communication device 60 (e.g., Bluetooth device, Near-Field Communication (NFC) device, RFID, WIFI antenna, and the like) that is coupled to the controller 24. The controller processor may be programmed to operate the wireless communication device 60 to wirelessly transmit information associated with the measured air pressure to a user device such as, for example, a smartphone or tablet computer.

In the illustrated embodiment, the lighted air supply delivery assembly 11 includes a main housing 62 that includes a housing outer surface 64 that defines a substantially rectangular shape. The main housing 62 includes a main housing body 66 having a housing inner surface 68 defining an equipment cavity 70, and a removable cover assembly 72 that is removable coupled to the main housing body 66 to enclose the equipment cavity 70 therebetween. The equipment cavity 70 is sized and shaped for housing the controller 24 and fiber-optic light engine 58 therein. The equipment cavity 70 may also house additional components such as, for example, the wireless communication device 60, the air pressure sensor 18, power supply circuit, additional sensors, and/or any other components of the lighted air supply delivery assembly 11.

The main housing body 66 also includes an interior wall 74 positioned within the equipment cavity 70 that includes an interior surface that defines an air sampling chamber 76 that is configured to receive oxygenated air from the oxygen concentrator 12. A sampling port 78 is defined along the interior wall 74 and is coupled in fluid communication with the air sampling chamber 76. A sampling tube 80 is coupled between the sampling port 78 and the air pressure sensor 18 to couple the air pressure sensor 18 in flow communication with the air sampling chamber 76 to enable the air pressure sensor 18 to sense and/or measure the air pressure within the air sampling chamber 76.

The main housing body 66 also includes an air inlet port 82 that is defined along the housing outer surface 64 and coupled in fluid communication with the air sampling chamber 76, and an air outlet port 84 that is defined along the housing outer surface 64 and coupled in fluid communication with the air sampling chamber 76. The air inlet port 82 is spaced a distance from the air outlet port 84. The lighted air supply delivery assembly 11 includes an air inflow tubing 86 that is coupled between the oxygen concentrator 12 and the air inlet port 82 for channeling oxygenated air from the oxygen concentrator 12 into the air sampling chamber 76. The air/light connection tubing 52 is coupled in fluid communication with the air sampling chamber 76 via the air outlet port 84 for channeling oxygenated air from the air sampling chamber 76 to the patient oxygen delivery assembly 20. The main housing body 66 also includes a lighting port 88 that is defined along the housing outer surface 64. The fiber-optic light engine 58 is positioned within the equipment cavity 70 and coupled to the lighting port 88.

In the illustrated embodiment, the lighted air supply delivery assembly 11 includes an air outflow and lighting assembly 90 that is coupled to the air outlet port 84, the lighting port 88, and the air/light connection tubing 52. The air outflow and lighting assembly 90 includes a fiber connector 92 that is removably coupled to the fiber-optic light engine 58 via the lighting port 88 and an air/light connection tee 94. In some embodiments, the fiber connector 92 includes a type ST fiber connector.

The air/light connection tee 94 includes an air/light outflow connector 96 that is coupled to the fiber connector 92 and includes an inner surface defining a second airflow chamber 54 and one or more second optical fibers 56 positioned within the second airflow chamber 54 for receiving light from the fiber-optic light engine 58 via the fiber connector 92. The air/light connection tee 94 also includes an outflow air supply connector 98 that extends outwardly from the air/light outflow connector 96. The outflow air supply connector 98 is adapted to be removably coupled to the air outlet port 84 for channeling oxygenated air from the air sampling chamber 76 into the second airflow chamber 54 of the air/light outflow connector 96.

The air/light connection tubing 52 is coupled to the air/light outflow connector 96 such that the second airflow chamber of the air/light outflow connector 96 is in fluid communication with the airflow chamber of the air/light connection tubing 52 for channeling oxygenated air from the air sampling chamber 76 to the patient oxygen delivery assembly 20 through the air/light connection tubing 52. In addition, the second optical fibers of the air/light outflow connector 96 are aligned with and contact the optical fibers of the air/light connection tubing 52 to facilitate transferring light from the fiber-optic light engine 58 through the optical fibers of the air/light connection tubing 52.

In this way, the controller 24 may monitor the air pressure within the patient oxygen delivery system 10 with the air pressure sensor 18 and operate the fiber-optic light engine 58 to display a flashing light that is visible through the air/light connection tubing 52 and transmit a wireless signal to a care giver's smartphone or tablet computer to notify the care giver of the reduction and/or loss of air pressure in the patient oxygen delivery system 10.

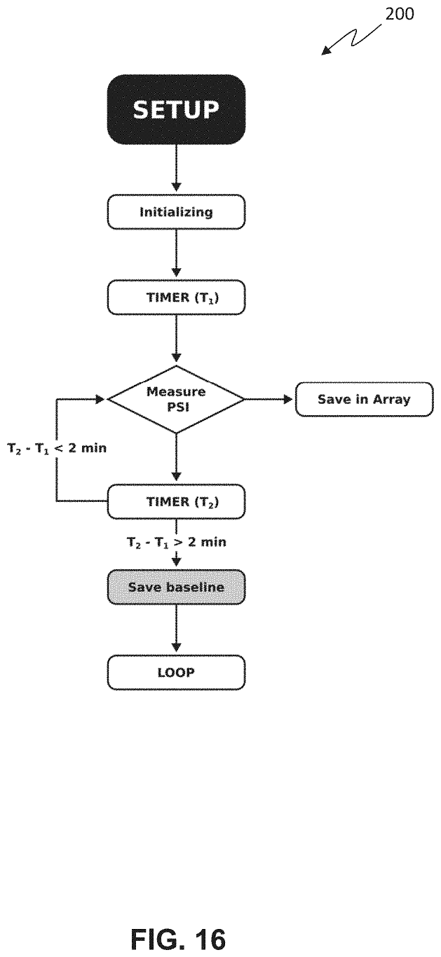

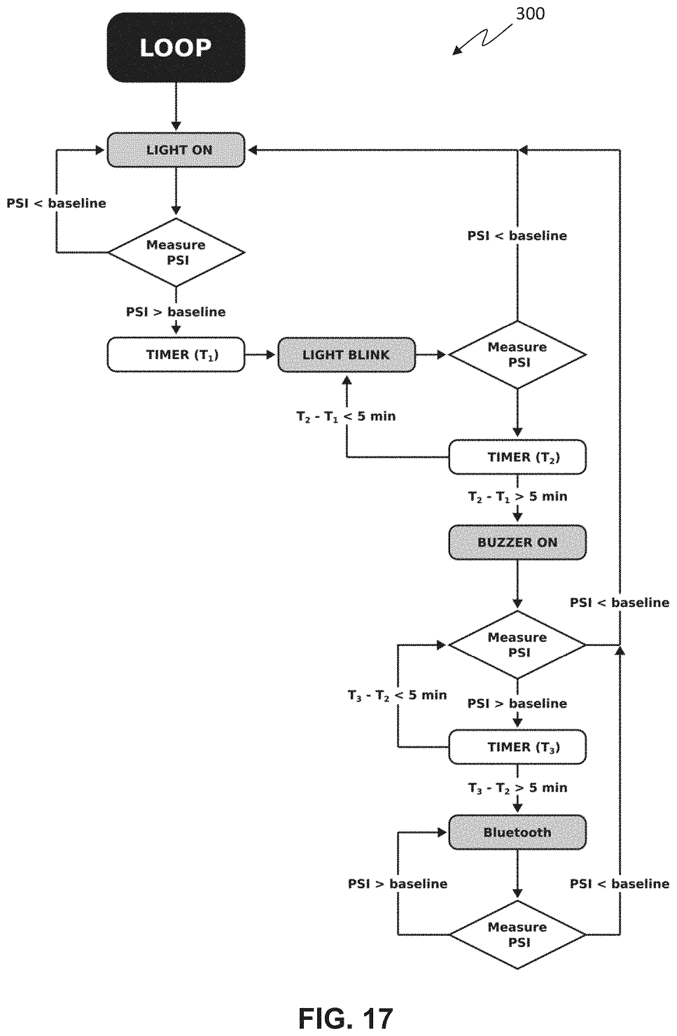

In some embodiments, the processor 24 is programmed to execute algorithms 200 and 300 shown in FIGS. 16 and 17. The algorithms include a plurality of steps. Each method step may be performed independently of, or in combination with, other method steps. Portions of the methods may be performed by any one of, or any combination of, the components of the system 10. For example, the processor 24 may be programmed to execute algorithm 200 to perform an initial set-up procedure to establish a baseline operating air pressure for use in operating the lighted air supply delivery assembly 11 by operating sensor 18 to measure and record the air pressure within the air sampling chamber 76 over a predefined period of time, and to execute algorithm 300 to operate the lighted air supply delivery assembly 11 using the established baseline operating air pressure.

Several configurations have been discussed in the foregoing description. However, the configurations discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

A controller, computing device, server or computer, such as described herein, includes at least one or more processors or processing units and a system memory (see above). The controller typically also includes at least some form of computer readable media. By way of example and not limitation, computer readable media may include computer storage media and communication media. Computer storage media may include volatile and nonvolatile, removable and non-removable media implemented in any method or technology that enables storage of information, such as computer readable instructions, data structures, program modules, or other data. Communication media typically embody computer readable instructions, data structures, program modules, or other data in a modulated data signal such as a carrier wave or other transport mechanism and include any information delivery media. Those skilled in the art should be familiar with the modulated data signal, which has one or more of its characteristics set or changed in such a manner as to encode information in the signal. Combinations of any of the above are also included within the scope of computer readable media.

The above description of illustrated examples of the present invention, including what is described in the Abstract, are not intended to be exhaustive or to be limitation to the precise forms disclosed. While specific embodiments of, and examples for, the invention are described herein for illustrative purposes, various equivalent modifications are possible without departing from the broader spirit and scope of the present invention.

What is claimed is:

1. A lighted air supply delivery assembly for use in delivering air to a patient, comprising:
an air/light connection tubing coupled between an oxygen concentrator and a patient oxygen delivery, the air/light connection tubing defining an airflow chamber for delivering a flow of oxygenated air from the oxygen concentrator to the patient oxygen delivery assembly, and including one or more optical fibers positioned within the airflow chamber;
a fiber-optic light engine coupled to the one or more optical fibers; and
a controller including a processor coupled to the fiber-optic light engine, the processor programmed to execute algorithm steps of:
operating the fiber-optic light engine to display a light visible through the air/light connection tubing via the one or more optical fibers.

2. The lighted air supply delivery assembly of claim 1, including:
an air pressure sensor for measuring an air pressure within the lighted air supply delivery assembly, the processor programmed to execute algorithm steps of:
operating the fiber-optic light engine to display the light visible through the air/light connection tubing based on the measured air pressure.

3. The lighted air supply delivery assembly of claim 2, including a wireless communication device coupled to the controller, the processor programmed to operate the wireless communication device to wirelessly transmit information associated with the measured air pressure.

4. The lighted air supply delivery assembly of claim 2, including:
a housing including:
a housing inner surface and a housing outer surface, the housing inner surface defining an equipment cavity for storing the controller and fiber-optic light engine therein;
an interior wall positioned within the equipment cavity and having an interior surface defining an air sampling chamber configured to receive oxygenated air from the oxygen concentrator; and
a sampling port defined along the interior wall and coupled in fluid communication with the air sampling chamber;
wherein the air pressure sensor is positioned within the equipment cavity and coupled to the sampling port for measuring air pressure within the air sampling chamber.

5. The lighted air supply delivery assembly of claim 4, wherein the housing includes an air outlet port defined along the housing outer surface and coupled in fluid communication with the air sampling chamber, the air/light connection tubing is coupled in fluid communication with the air sampling chamber via the air outlet port for channeling oxygenated air from the air sampling chamber to the patient oxygen delivery assembly.

6. The lighted air supply delivery assembly of claim 5, including the housing including an air inlet port defined along the housing outer surface and coupled in fluid communication with the air sampling chamber; and
an air inflow tubing coupled between the oxygen concentrator and the air inlet port for channeling oxygenated air from the oxygen concentrator into the air sampling chamber.

7. The lighted air supply delivery assembly of claim 6, wherein the housing includes:
a lighting port defined along the housing outer surface;
the fiber-optic light engine positioned within the equipment cavity and coupled to the lighting port.

8. The lighted air supply delivery assembly of claim 7, including:
an air outflow and lighting assembly coupled to the air outlet port and the lighting port, the air outflow and lighting assembly including:
a fiber connector removably coupled to the fiber-optic light engine via the lighting port; and
an air/light connection tee including:
an air/light outflow connector coupled to the fiber connector and including an inner surface defining a second airflow chamber and one or more second optical fibers positioned within the second airflow chamber for receiving light from the fiber-optic light engine via the fiber connector;
an outflow air supply connector extending outwardly from the air/light outflow connector and removably coupled to the air outlet port for channeling oxygenated air from the air sampling chamber into the second airflow chamber;
the air/light connection tubing is coupled to the air/light outflow connector such that the second airflow chamber of the air/light outflow connector is in fluid communication with the airflow chamber of the air/light connection tubing for channeling oxygenated air from the air sampling chamber to the patient oxygen delivery assembly, and the one or more second optical fibers of the air/light outflow connector contact the one or more optical fibers of the air/light connection tubing to transfer light from the fiber-optic light engine through the one or more optical fibers of the air/light connection tubing.

9. The lighted air supply delivery assembly of claim 8, wherein the fiber connector includes an ST fiber connector.

10. An oxygen delivery system for delivering oxygenated air to a patient comprising:
an oxygen concentrator;
a patient oxygen delivery assembly adapted to be mounted to a patient's face; and
a lighted air supply delivery assembly coupled between the oxygen concentrator and the patient oxygen delivery assembly, the lighted air supply delivery assembly including:
an air/light connection tubing defining an airflow chamber for delivering a flow of oxygenated air from the oxygen concentrator to the patient oxygen delivery assembly, and including one or more optical fibers positioned within the airflow chamber;
a fiber-optic light engine coupled to the one or more optical fibers; and
a controller including a processor coupled to the fiber-optic light engine, the processor programmed to execute algorithm steps of:
operating the fiber-optic light engine to display a light visible through the air/light connection tubing via the one or more optical fibers.

11. The oxygen delivery system of claim 10, wherein the lighted air supply delivery assembly includes:
an air pressure sensor for measuring an air pressure within the lighted air supply delivery assembly, the processor programmed to execute algorithm steps of:
operating the fiber-optic light engine to display the light visible through the air/light connection tubing based on the measured air pressure.

12. The oxygen delivery system of claim 11, wherein the lighted air supply delivery assembly includes a wireless communication device coupled to the controller, the processor programmed to operate the wireless communication device to wirelessly transmit information associated with the measured air pressure.

13. The oxygen delivery system of claim 11, wherein the lighted air supply delivery assembly includes:
a housing including:
a housing inner surface and a housing outer surface, the housing inner surface defining an equipment cavity for storing the controller and fiber-optic light engine therein;
an interior wall positioned within the equipment cavity and having an interior surface defining an air sampling chamber configured to receive oxygenated air from the oxygen concentrator; and
a sampling port defined along the interior wall and coupled in fluid communication with the air sampling chamber;
wherein the air pressure sensor is positioned within the equipment cavity and coupled to the sampling port for measuring air pressure within the air sampling chamber.

14. The oxygen delivery system of claim 13, wherein the housing includes an air outlet port defined along the housing outer surface and coupled in fluid communication with the air sampling chamber, the air/light connection tubing is coupled in fluid communication with the air sampling chamber via the air outlet port for channeling oxygenated air from the air sampling chamber to the patient oxygen delivery assembly.

15. The oxygen delivery system of claim 14, wherein the housing includes:
an air inlet port defined along the housing outer surface and coupled in fluid communication with the air sampling chamber;
the lighted air supply delivery assembly includes an air inflow tubing coupled between the oxygen concentrator and the air inlet port for channeling oxygenated air from the oxygen concentrator into the air sampling chamber.

16. The oxygen delivery system of claim 15, wherein the housing includes a lighting port defined along the housing outer surface, the fiber-optic light engine is positioned within the equipment cavity and coupled to the lighting port.

17. The oxygen delivery system of claim 16, wherein the lighted air supply delivery assembly includes:
an air outflow and lighting assembly coupled to the air outlet port and the lighting port, the air outflow and lighting assembly including:
a fiber connector removably coupled to the fiber-optic light engine via the lighting port; and
an air/light connection tee including:
an air/light outflow connector coupled to the fiber connector and including an inner surface defining a second airflow chamber and one or more second optical fibers positioned within the second airflow chamber for receiving light from the fiber-optic light engine via the fiber connector;
an outflow air supply connector extending outwardly from the air/light outflow connector and removably coupled to the air outlet port for channeling oxygenated air from the air sampling chamber into the second airflow chamber;
the air/light connection tubing is coupled to the air/light outflow connector such that the second airflow chamber of the air/light outflow connector is in fluid communication with the airflow chamber of the air/light connection tubing for channeling oxygenated air from the air sampling chamber to the patient oxygen delivery assembly, and the one or more second optical fibers of the air/light outflow connector contact the one or more optical fibers of the air/light connection tubing to transfer light from the fiber-optic light engine through the one or more optical fibers of the air/light connection tubing.

18. The oxygen delivery system of claim 17, wherein the fiber connector includes an ST fiber connector.

19. The oxygen delivery system of claim 10, wherein the patient oxygen delivery assembly includes at least one of a nasal cannula and a face mask.

20. A method of assembling a lighted air supply delivery assembly for use in delivering air to a patient, the method including:
providing an air/light connection tubing defining an airflow chamber for delivering a flow of oxygenated air from an oxygen concentrator to a patient oxygen delivery assembly, the air/light connection tubing including one or more optical fibers positioned within the airflow chamber;
coupling a fiber-optic light engine to the one or more optical fibers;
coupling an air pressure sensor for measuring an air pressure to the an air/light connection tubing; and
coupling a controller to the fiber-optic light engine and the air pressure sensor, the controller including a processor programmed to execute algorithm steps of:
operating the fiber-optic light engine to display the light visible through the air/light connection tubing based on the measured air pressure.
